(19) United States
(12) Patent Application Publication
(10) Pub. No.: US 2017/0198575 A1
(43) Pub. Date: Jul. 13, 2017

(54) Asphaltene Concentration Analysis Via NMR

(71) Applicant: Halliburton Energy Services, Inc., Houston, TX (US)

(72) Inventors: Magdalena Traico Sandor, Humble, TX (US); Songhua Chen, Katy, TX (US)

(73) Assignee: Halliburton Energy Services, Inc., Houston, TX (US)

(21) Appl. No.: 15/319,989

(22) PCT Filed: Aug. 18, 2015
(86) PCT No.: PCT/US15/45602

§ 371 Date: Dec. 19, 2016

(51) Int. Cl. E21B 49/08; G01N 33/28; E21B 37/06; G01N 24/08

(52) U.S. Cl. CPC E21B 49/08; G01N 24/081; G01N 33/2835; G01N 33/2823; E21B 37/06; E21B 2049/085; E21B 47/12

(57) ABSTRACT

Analyzing crude oils and, more specifically, indirectly measuring asphaltene concentration in crude oils may be performed via nuclear magnetic resonance (NMR) techniques. For example, determining the asphaltene concentration of a crude oil sample having an unknown concentration of asphaltene and having the API gravity of about 20 to about 41 may be achieved by applying a measured NMR property of the unknown sample to a mathematical regression for asphaltene concentration in crude oil as a function of an NMR property according to the following equation where C is the asphaltene concentration, k is the Huggins constant that describes the solvent quality, [η] is the intrinsic viscosity.

$$\frac{1}{{T}_{1}} = \frac{1}{{T}_{2}} ∼ 1 + [ η ] C + {k [ η ]}^{2} {C}^{2}$$

### BACKGROUND

The present application relates to analyzing crude oil and the like with nuclear magnetic resonance (NMR) techniques.

Crude oil is composed primarily of four types of hydrocarbons: saturates (primarily non-polar straight hydrocarbons, branched chain hydrocarbons, and cyclic paraffins), aromatics (including fused benzene rings compounds), resins (polar aromatic rings systems containing nitrogen, oxygen, or sulfur), and asphaltenes (highly polar, complex aromatic ring compounds with varying composition, containing nitrogen, oxygen, and sulfur). The saturates, aromatics, and resins are sometimes collectively referred to as maltenes. The asphaltene fraction of crude oil is defined as the portion that is not soluble in straight-chain solvents such as pentane or heptane. Generally, asphaltenes exist as a colloidal suspension stabilized by maltenes (especially, resins).

When producing crude oil from a subterranean formation, asphaltenes and some resins (e.g., paraffins) may build up and deposit in the formation (e.g., on fracture faces and in formation pores) and in tubulars, production equipment, storage equipment, transportation equipment, and related apparatus. These deposits may reduce fluid flow and, consequently, decrease oil production.

In general, deposits with high concentrations of asphaltene are hard and brittle while deposits formed primarily of paraffinic compounds are soft and pliable. Thus, deposits containing asphaltenes are typically more troublesome because mechanical methods and conventional solvents are relatively ineffective in their removal. However, if the asphaltene concentration can be ascertained prior to or monitored during crude oil production, the production operations may be performed in ways that reduce the formation of asphaltene deposits.

Additionally, crude oil refining typically separates crude oil into its individual components. The refining processes implemented are, to some degree, defined by the concentration of each of the crude oil components. Therefore, asphaltene concentration is also important in refining operations.

Currently, saturate/aromatic/resin/asphaltene (SARA) analysis is one of the methods used to ascertain asphaltene concentration. SARA analysis uses solubility of the crude oil components in various solvents to physically separate each of the crude oil components. SARA analysis is an extensive test that is performed in a laboratory. Accordingly, current techniques do not readily allow for monitoring asphaltene concentration during crude oil production or refining. Additionally, ascertaining asphaltene concentration prior to production operation may be time consuming because samples are first retrieved then sent to another location for detailed analysis.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates an exemplary drilling assembly for implementing the NMR analysis methods described herein.

FIG. 2 illustrates a wireline system suitable for implementing the NMR analysis methods described herein.

FIG. 3 illustrates a hydrocarbon production system suitable for implementing the NMR analysis methods described herein.

FIG. 4 illustrates a relationship between asphaltene concentration (weight fraction) versus 1/T1GM (spin-lattice relaxation (T1) geometric mean (GM)) and 1/T2GM (spin-spin relaxation (T2) GM) of the crude oil samples.

### DETAILED DESCRIPTION

The present application relates to analyzing crude oils and, more specifically, indirectly measuring asphaltene concentration in crude oils with NMR techniques using a correlation (e.g., a mathematical regression, a graph, or the like) of asphaltene concentration in crude oil as a function of an NMR property. By using NMR techniques, a physical separation of the crude oil components is not required, which provides a more time efficient and cost effective analysis technique. Additionally, the hardware needed for the analysis may be readily adapted to be used in the field or in a refinery for monitoring asphaltene concentration in crude oil.

NMR relaxation of fluids is sensitive to a range of molecular motions, which is related to the size distribution of molecules and their intermolecular and intramolecular interactions. The present application extends NMR sensitivity to include the saturate, aromatic, resin, and asphaltene (SARA) components of crude oil from a subterranean formation, which have a wide range in molecular weights, aromaticity, and polarity.

Asphaltenes are the heaviest and most polar molecules in crude oil from subterranean formation. Asphaltenes are also porous macromolecules that are highly susceptible to aggregation and flocculation. These complex molecules readily form micelle structures that are capable of stable suspension in crude oil through solvation with high polarity molecules such as resins and aromatics. Additionally, the micelle structures may be further stabilized by adsorption of resin molecules by acting as a transition barrier between polar and non-polar components of the crude oil. The porous structure and strong polarity of asphaltene micelles, therefore, permits coupling of varying degrees of strength between the molecular motions of asphaltenes and other constituents of crude oil having compatible solubility. In some instances, because porous asphaltene micelles are needed for the NMR analyses described herein (e.g., application of Equation 3 described further herein), the crude oil samples may be limited to a American Petroleum Institute gravity (API gravity) of about 20° to about 41°. Generally, higher API gravity samples have too little asphaltene to produce asphaltene micelles, and lower API gravity samples have so much asphaltene that the asphaltene micelles aggregate into a different form.

However, coupling between asphaltene and saturates is much weaker in comparison and more likely to occur indirectly between saturates and neutral resins or lighter aromatic molecules. In crude oil, short range dipole-dipole interactions between spins contribute significantly to the local field fluctuations and energy dispersion that govern spin-spin relaxation (T2) and spin-lattice relaxation (T1), respectively. For crude oil of very low viscosity, these dipolar fields between neighboring spins are averaged out through fast molecular motions causing a T2 as long as T1. However, this may not be true for high molecular weight components of the crude oil, whereby the molecular motion may be significantly slower or partially immobilized due to entanglement.

Through translational and rotational diffusion of molecules, collisions cause nuclear spins to relax by both intermolecular and intramolecular dipole-dipole interactions. Due to the difficulty in separating these contributions, the intramolecular interaction due to sensitive dependence on spin distance are mainly considered in the present application. Under the assumption that fast motion limits the T2, the T1 can be expressed as Equation 1, where kT is the thermal energy, a is the hydrodynamic radius of the molecule, and η is the viscosity.

$$\frac{1}{{T}_{1}} = \frac{1}{{T}_{2}} ∼ {a}^{3} η / kT \qquad Equation 1$$

Therefore, the total T2 relaxation rate measured by NMR may have additive contributions from each SARA component in crude oil. However, due to the inherent complexity of the crude oil, it is difficult to quantify independently each component's contribution to the total relaxation. Since asphaltenes represent the heaviest and most polar components of crude oil, a more simplified approach would be to consider just the effect of asphaltene concentration to the total relaxation. Therefore, in the fast diffusion regime, the total relaxation rate can be considered asphaltene-induced relaxation, R_{1,2,asphaltene}.

For asphaltene-induced relaxation, components of crude oil that are directly associated with asphaltene aggregates temporarily share the same axis of rotation, which would give rise to a larger effective hydrodynamic radius and, thus, larger relaxation rate. Due to the porous structure and fractal nature of asphaltene aggregates, other crude oil components (e.g., resins, asphaltene nano-aggregates, and aromatics) that diffuse through the porous asphaltene aggregates may be partially immobilized, entangled, and even relax due to locally residing paramagnetic impurities. On the other hand, saturates, the lightest and lowest polarity components of crude oil, may have the weakest interatomic interactions with the asphaltene aggregates. However, saturates may come within close proximity to asphaltenes on the timescale of the NMR experiments through diffusion.

Neutron scattering studies have demonstrated that as the asphaltene concentration or volume fraction is increased in crude oil, the aggregates change in number, size, and structure, which is directly related to the fractal dimension. This contributes significantly to the hydrodynamic volume and viscous drag forces, which effects T1 and T2. The fractal dimension has demonstrated a significant role in describing the aggregated asphaltene structures. For example, the fractal dimension may range from about 3 in the dilute limit to about 1.8 at high concentrations (e.g., 40-60% wt.). As a result, relationships between average weight, radii of gyration, and volume concentration take power scaling laws where the exponent is related to the fractal dimension.

However, asphaltenes have been demonstrated to play an even more influential role in rheological measurements giving rise to an exponential increase in viscosity with a minor increase in asphaltene concentration. The concentration regimes of asphaltenes may vary from dilute with a linear viscosity dependence to semi-dilute (e.g., about 10 wt. %) where the viscosity takes on an exponential dependence. A colloidal description is often used to characterize the relative viscosity, defined as the ratio of the viscosity of the solution to the viscosity of the solvent, of asphaltenes in various solvents and can be expressed as Equation 2, where C is the asphaltene concentration, k is the Huggins constant that describes the solvent quality, [η] is the intrinsic viscosity (which is a measure of the solutes or asphaltene's ability to increase the solvent viscosity), and ηᵣ is the relative viscosity, which can be determined by computing the ratio of the solution viscosity to solvent viscosity.

$$\frac{{η}_{r} - 1}{C} = [ η ] + {k [ η ]}^{2} C \qquad Equation 2$$

Assuming the maltene components of a crude oil represent the solvent, Equation 2 can be generalized for describing the T2 and T1 dependence on asphaltene weight fraction as Equation 3, where [η] and k may be measured or estimated. [η] and k may be determined experimentally by plotting asphaltene concentration (weight fraction) as a function of 1/T₁ or 1/T₂ (i.e., C(1/T₁) or C(1/T₂). Example 1 below provides an example of experimentally obtaining the [η] and k.

$$\frac{1}{{T}_{1}} = \frac{1}{{T}_{2}} ∼ 1 + [ η ] C + {k [ η ]}^{2} {C}^{2} \qquad Equation 3$$

Therefore, methods described herein may, in some instances, involve deriving Equation 3 using crude oil samples with known asphaltene concentrations. Then, T1 or T2 may be measured for other crude oil samples having unknown asphaltene concentrations (referred to herein as “unknown samples”). The measured T1 or T2 may be used in the derived Equation 3 to determine the asphaltene concentration in the unknown samples.

In some instances, the T2 may be determined using a Carr-Meiboom-Purcell-Gill (CMPG) pulse sequence, where T2 is determined from the characteristic time of the CMPG echo train decay.

Because crude oil samples with an API gravity of about 20° to about 41° have similar T1 and T2 (e.g., a T1:T2 ratio of about 1 to about 1.5), the derivation of Equation 3 may be done using T1, T2, a CMPG echo train, or any combination thereof, and the analysis of the unknown samples may be done with the same or different NMR properties (i.e., T1, T2, a CMPG echo train, or any combination thereof). For example, T1 may be used to derive Equation 3 and as the NMR property measured when determining asphaltene concentration for the unknown sample. In another example, T1 may be used to derive Equation 3, and T2 may be used as the NMR property measured when determining asphaltene concentration for the unknown sample. In yet another example, two or more of T1, T2, and a CMPG echo train may be used to derive Equation 3, and one of T1, T2, and a CMPG echo train may be used as the NMR property measured when determining asphaltene concentration for the unknown sample.

In alternate embodiments, a correlation between an NMR property (e.g., the T1, T2, CMPG echo train, or any combination thereof) and asphaltene concentration may be derived by plotting a graphs and/or determining a trend line (or other mathematical regression) of the NMR property measured for the crude oil samples with known asphaltene concentrations as a function of asphaltene concentration. Then, the same NMR property may be measured for a sample having an unknown concentration of asphaltene where the correlation, the graph or a corresponding trend line, in this instance, may be used to determine the asphaltene concentration in the unknown sample. In instances, where the T1:T2 ratio of about 1 to about 1.5 as described above, a different NMR property may be measured for the unknown sample and applied to the derived correlation.

The foregoing methods may be applicable to wellbore operations (e.g., wireline logging operations, logging-while-drilling (LWD) operations, and production operations) and refining operation.

For example, in some embodiments, an NMR logging operation may measure the T1, T2, CMPG echo train, or any combination thereof of an unknown crude oil in a subterranean formation. The NMR property measured may be used in a correlation described herein (e.g., Equation 3, a graph, a trend line, or the like) to determine the asphaltene concentration of the unknown crude oil in the subterranean formation. Such NMR logging operations may be LWD operations where the NMR property is measured while drilling a wellbore penetrating a subterranean formation. Alternatively, the NMR logging operation may be a wireline operation where the NMR property is measured while conveying an NMR tool through a wellbore penetrating a subterranean formation.

In another example, a core sample that contains an unknown crude oil may be retrieved from a wellbore. The unknown crude oil may then be analyzed (e.g., at the well site or in a laboratory) via NMR and a correlation described herein to ascertain the asphaltene concentration of the unknown crude oil.

In yet another example, an unknown crude oil being produced from a subterranean formation may be sampled and analyzed via NMR and a correlation described herein to ascertain the asphaltene concentration of the unknown crude oil.

Depending on the asphaltene concentration, which may be ascertained by the foregoing examples, an operator may adjust the subsequent wellbore operations to mitigate the formation of asphaltene deposits (e.g., formed by asphaltene precipitation).

In some instances, the crude oil in the formation may be treated with a chemical that mitigates asphaltene precipitation (e.g., an asphaltene solvent, a dispersant, or the like).

In some instances, during a production operation, the temperature of the wellhead or other equipment may be elevated to mitigate temperature-initiated precipitation of asphaltene. Additionally or alternatively, chemicals that mitigate asphaltene precipitation (e.g., asphaltene solvents, dispersants, or the like) may be added to the crude oil at or near the wellhead to mitigate asphaltene precipitation.

In some instances, during a production operation, the crude oil may be treated with steam (e.g., via steam-assisted gravity drainage) to mitigate asphaltene precipitation. Additionally or alternatively, the crude oil in the formation may be treated with chemicals that mitigate asphaltene precipitation (e.g., asphaltene solvents, dispersants, or the like).

In some instances, after a production operation and before transporting the crude oil by pipeline, chemicals that mitigate asphaltene precipitation (e.g., asphaltene solvents, dispersants, or the like) may be added to the crude oil to mitigate asphaltene precipitation.

In refining operations, one or more crude oils may be used as feedstocks for the operation. In some instances, two or more feedstocks may be mixed to achieve a refining feedstock with desired asphaltene concentration for the refining operation parameters being implemented. The asphaltene concentration of the two or more feedstocks may be determined by NMR analysis and a correlation described herein to achieve the desired asphaltene concentration in the refining feedstock.

In alternate embodiments, the asphaltene concentration in the refining feedstock, which may be a single feedstock or a mixture of feedstocks, may be determined by NMR analysis and a correlation described herein. Then, the refining parameters (e.g., temperatures, pressures, etc.) may be adjusted based on the asphaltene concentration in the refining feedstock.

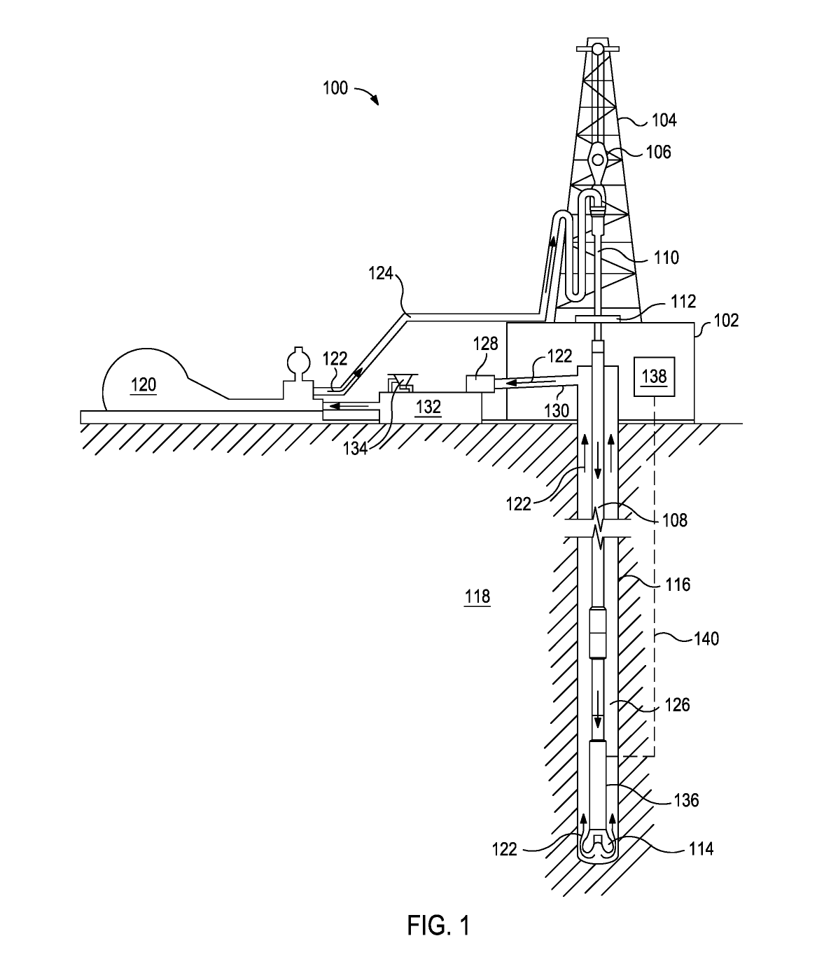

FIG. 1 illustrates an exemplary drilling assembly 100 for implementing the NMR analysis methods described herein. It should be noted that while FIG. 1 generally depicts a land-based drilling assembly, those skilled in the art will readily recognize that the principles described herein are equally applicable to subsea drilling operations that employ floating or sea-based platforms and rigs, without departing from the scope of the disclosure.

As illustrated, the drilling assembly 100 may include a drilling platform 102 that supports a derrick 104 having a traveling block 106 for raising and lowering a drill string 108. The drill string 108 may include, but is not limited to, drill pipe and coiled tubing, as generally known to those skilled in the art. A kelly 110 supports the drill string 108 as it is lowered through a rotary table 112. A drill bit 114 is attached to the distal end of the drill string 108 and is driven either by a downhole motor and/or via rotation of the drill string 108 from the well surface. As the bit 114 rotates, it creates a wellbore 116 that penetrates various subterranean formations 118. Along the drill string 108 logging while drilling (LWD) or measurement while drilling (MWD) equipment 136 is included.

In the present application, the LWD/MWD equipment 136 may be capable of NMR analysis of the subterranean formation 118 proximal to the wellbore 116. The LWD/MWD equipment 136 may transmit the measured data to a processor 138 at the surface wired or wirelessly. Transmission of the data is generally illustrated at line 140 to demonstrate communicable coupling between the processor 138 and the LWD/MWD equipment 136 and does not necessarily indicate the path to which communication is achieved.

A pump 120 (e.g., a mud pump) circulates drilling fluid 122 through a feed pipe 124 and to the kelly 110, which conveys the drilling fluid 122 downhole through the interior of the drill string 108 and through one or more orifices in the drill bit 114. The drilling fluid 122 is then circulated back to the surface via an annulus 126 defined between the drill string 108 and the walls of the wellbore 116. At the surface, the recirculated or spent drilling fluid 122 exits the annulus 126 and may be conveyed to one or more fluid processing unit(s) 128 via an interconnecting flow line 130. After passing through the fluid processing unit(s) 128, a “cleaned” drilling fluid 122 is deposited into a nearby retention pit 132 (i.e., a mud pit). While illustrated as being arranged at the outlet of the wellbore 116 via the annulus 126, those skilled in the art will readily appreciate that the fluid processing unit(s) 128 may be arranged at any other location in the drilling assembly 100 to facilitate its proper function, without departing from the scope of the scope of the disclosure.

Chemicals, fluids, additives, and the like may be added to the drilling fluid 122 via a mixing hopper 134 communicably coupled to or otherwise in fluid communication with the retention pit 132. The mixing hopper 134 may include, but is not limited to, mixers and related mixing equipment known to those skilled in the art. In other embodiments, however, the chemicals, fluids, additives, and the like may be added to the drilling fluid 122 at any other location in the drilling assembly 100. In at least one embodiment, for example, there could be more than one retention pit 132, such as multiple retention pits 132 in series. Moreover, the retention pit 132 may be representative of one or more fluid storage facilities and/or units where the chemicals, fluids, additives, and the like may be stored, reconditioned, and/or regulated until added to the drilling fluid 122.

The processor 138 may be a portion of computer hardware used to implement the various illustrative blocks, modules, elements, components, methods, and algorithms described herein. The processor 138 may be configured to execute one or more sequences of instructions, programming stances, or code stored on a non-transitory, computer-readable medium. The processor 138 can be, for example, a general purpose microprocessor, a microcontroller, a digital signal processor, an application specific integrated circuit, a field programmable gate array, a programmable logic device, a controller, a state machine, a gated logic, discrete hardware components, an artificial neural network, or any like suitable entity that can perform calculations or other manipulations of data. In some embodiments, computer hardware can further include elements such as, for example, a memory (e.g., random access memory (RAM), flash memory, read only memory (ROM), programmable read only memory (PROM), erasable read only memory (EPROM)), registers, hard disks, removable disks, CD-ROMS, DVDs, or any other like suitable storage device or medium.

Executable sequences described herein can be implemented with one or more sequences of code contained in a memory. In some embodiments, such code can be read into the memory from another machine-readable medium. Execution of the sequences of instructions contained in the memory can cause a processor 138 to perform the process steps described herein. One or more processors 138 in a multi-processing arrangement can also be employed to execute instruction sequences in the memory. In addition, hard-wired circuitry can be used in place of or in combination with software instructions to implement various embodiments described herein. Thus, the present embodiments are not limited to any specific combination of hardware and/or software.

As used herein, a machine-readable medium will refer to any medium that directly or indirectly provides instructions to the processor 138 for execution. A machine-readable medium can take on many forms including, for example, non-volatile media, volatile media, and transmission media. Non-volatile media can include, for example, optical and magnetic disks. Volatile media can include, for example, dynamic memory. Transmission media can include, for example, coaxial cables, wire, fiber optics, and wires that form a bus. Common forms of machine-readable media can include, for example, floppy disks, flexible disks, hard disks, magnetic tapes, other like magnetic media, CD-ROMs, DVDs, other like optical media, punch cards, paper tapes and like physical media with patterned holes, RAM, ROM, PROM, EPROM and flash EPROM.

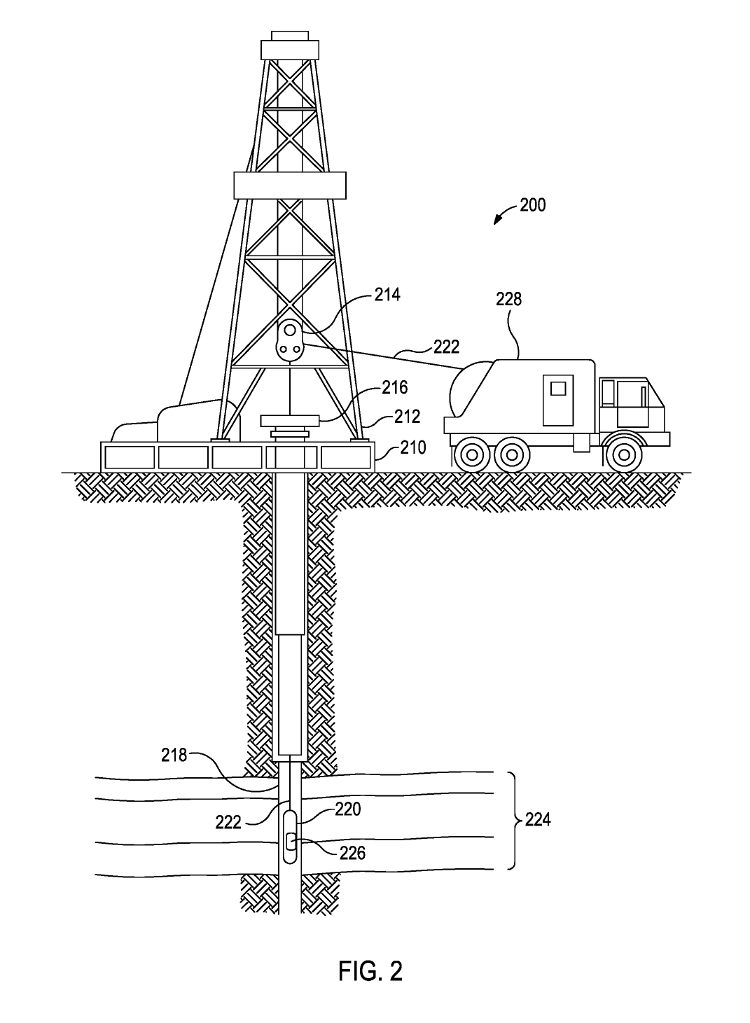

FIG. 2 illustrates a wireline system 200 suitable for implementing the NMR analysis methods described herein. As illustrated, a drilling platform 210 may be equipped with a derrick 212 that supports a hoist 214. Drilling oil and gas wells are commonly carried out using a string of drill pipes connected together so as to form a drilling string that is lowered through a rotary table 216 into a wellbore 218. Here, it is assumed that the drilling string has been temporarily removed from the wellbore 218 to allow an NMR logging tool 220 to be lowered by wireline or logging cable 222 into the wellbore 218. Typically, the NMR logging tool 220 is lowered to a region of interest and subsequently pulled upward at a substantially constant speed. During the upward trip, instruments included in the NMR logging tool 220 may be used to perform measurements on the subterranean formation 224 adjacent the wellbore 218 as the NMR logging tool 220 passes by. The NMR relaxation data may be communicated to a logging facility 228 for storage, processing, and analysis. The logging facility 228 may be provided with electronic equipment like processors described above for various types of signal processing.

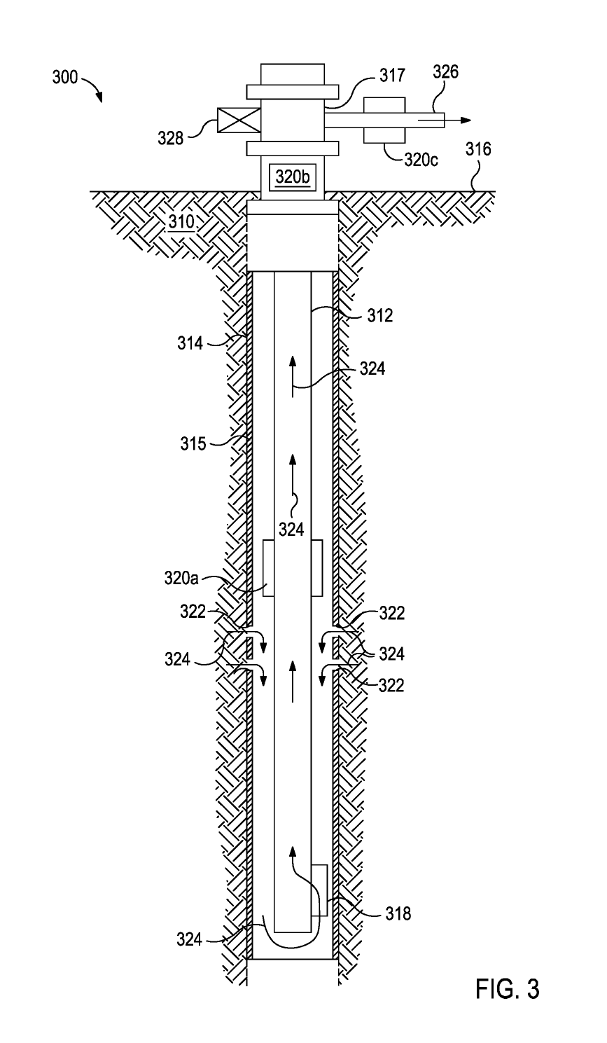

FIG. 3 illustrates a hydrocarbon production system 300 suitable for implementing the NMR analysis methods described herein. The system 300 may include a tubular 312 disposed in a wellbore 314 that penetrates a subterranean formation 310, and the tubular 312 may be adapted to convey fluids from the subterranean formation 310 to a surface location 316 in the direction generally indicated by arrows 324. A downhole fluid lift system 318, operable to lift fluids towards the surface location 316, is at least partially disposed in the wellbore 314 and may be integrated into, coupled to, or otherwise associated with the tubular 312.

The tubular 312 may be an appropriate tubular completion member configured for transporting fluids. For example, the tubular 312 may be jointed production tubing, coiled tubing, production tubing, or similar pipe lengths.

A wellhead 317 may be disposed proximal to the surface location 316. The wellhead 317 may be operatively coupled to a casing 315 that extends a substantial portion of the length of the wellbore 314 surface location towards the subterranean formation 310. In some instances, the casing 315 may terminate at or above one of the subterranean formation 310, thereby leaving the wellbore 314 un-cased through the subterranean formation 310, which is commonly referred to as “open hole.” In other instances, as illustrated, the casing 315 may extend through the subterranean formation 310 and may include apertures 322 either formed prior to installing the casing 315 or otherwise by downhole perforating operations to allow fluid communication between the interior of the wellbore 314 and the subterranean formation 310. Some, all, or none of the casing 315 may be affixed to the adjacent ground material with a cement jacket or the like.

One or more NMR tools 320 may be included in the system 300. As illustrated, the system 300 includes three NMR tools 320a,320b,320c. A first NMR tool 320a is coupled to or otherwise a portion of the tubular 312. This NMR tool 320a may be configured for measuring one or more NMR properties of the surrounding subterranean formation 310, one or more NMR properties of a fluid in the tubular 312, or both. A second NMR tool 320b is illustrated as coupled to or otherwise a portion of the wellhead 317 for measuring one or more NMR properties of a fluid passing therethrough. A third NMR tool 320c is illustrated as coupled to or otherwise a portion of a pipe 326 or other tubular extending from the wellhead 317 for measuring one or more NMR properties of a fluid passing therethrough. The NMR tools 320a,320b,320c may measure fluids as described in the formation 310, tubular 312, wellhead, or pipe 326. Alternatively, system 300 may be configured for a fluid bypass to flow through one or more of the NMR tools 320a,320b,320c for measuring NMR properties of the fluid. Such a bypass may facilitate connecting and disconnecting the NMR tools 320a,320b,320c to the system 300 as needed for measuring or maintenance.

The system 300 may also further include a control system(s) 328 with a processor (e.g., similar to processor 138 described above) communicably coupled to various components of the system 300 (e.g., the downhole fluid lift system 318, the NMR tools 320a,320b,320c, and the like) and be capable of executing the mathematical algorithms, methods, and analyses described herein.

In each of the foregoing systems 100,200,300 of FIGS. 1-3, wellbore 116,218,314 is a substantially vertical wellbore extending from a surface location into the subterranean formation. However, the systems and methods described herein can also be used with other wellbore configurations (e.g., deviated wellbores, horizontal wellbores, multilateral wellbores, and other configurations).

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating the invention embodiments disclosed herein are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

Embodiments disclosed herein include Embodiment A, Embodiment B, and Embodiment C.

Embodiment A is a method that includes determining a correlation for asphaltene concentration in crude oil as a function of a NMR property using a plurality of crude oil samples having a known concentration of asphaltene, wherein the first NMR property is a spin-spin relaxation (T2), a spin-lattice relaxation (T1), a Carr-Meiboom-Purcell-Gill (CMPG) echo train, or any combination thereof; measuring a second NMR property of a crude oil sample having an unknown concentration of asphaltene, wherein the second NMR property includes the T1, the T2, the CMPG echo train, or any combination thereof; and determining the asphaltene concentration of the crude oil sample having the unknown concentration of asphaltene by applying the second NMR property to the correlation.

Embodiment A may have one or more of the following additional elements in any combination: Element A1: wherein the correlation is a mathematical regression according to Equation 3 and the plurality of crude oil samples having the known concentration of asphaltene have an American Petroleum Institute (API) gravity of about 20 to about 41, wherein C is an asphaltene concentration in the plurality of crude oil samples having the known concentration of asphaltene, k is a Huggins constant that describes solvent quality of the plurality of crude oil samples having the known concentration of asphaltene, and [η] is an intrinsic viscosity of the plurality of crude oil samples having the known concentration of asphaltene; Element A2: wherein the first NMR property and the second NMR property are different; Element A3: wherein the plurality of crude oil samples having the known concentration of asphaltene have an API gravity of about 20 to about 41; Element A4: the method further including conveying an NMR tool via wireline through a wellbore penetrating a subterranean formation while measuring the second NMR property, wherein the crude oil sample having the unknown concentration of asphaltene is a crude oil in the subterranean formation surrounding the wellbore; Element A5: the method further including drilling a wellbore penetrating a subterranean formation while measuring the second NMR property, wherein the crude oil sample having the unknown concentration of asphaltene is a crude oil in the subterranean formation surrounding the wellbore; Element A6: the method further including Element A4 and/or Element A5 and treating a crude oil in the subterranean formation with a chemical to reduce asphaltene precipitation; and producing the crude oil in the subterranean formation; Element A7: the method further including Element A4 and/or Element A5 and treating a crude oil in the subterranean formation with steam; and producing the crude oil in the subterranean formation; Element A8: wherein the crude oil sample having the unknown concentration of asphaltene is a refinery feedstock; Element A9: wherein the crude oil sample having the unknown concentration of asphaltene is a first feedstock, the method further including: mixing the first feedstock with a second feedstock of crude oil with a known concentration of asphaltene in appropriate quantities to produce a refinery feedstock with a desired asphaltene concentration; Element A10: the method further including producing a crude oil from a subterranean formation using a system that includes a wellhead, wherein the crude oil sample having the unknown concentration of asphaltene is a portion of the crude oil; then, measuring the second NMR property of the crude oil sample having the unknown concentration of asphaltene; and treating the crude oil at or near a wellhead with a chemical to reduce asphaltene precipitation; and Element A11: the method further including producing a crude oil from a subterranean formation using a system that includes a wellhead, wherein the crude oil sample having the unknown concentration of asphaltene is a portion of the crude oil; then, measuring the second NMR property of the crude oil sample having the unknown concentration of asphaltene; and changing a temperature at the wellhead to reduce asphaltene precipitation.

By way of non-limiting example, exemplary combinations applicable to Embodiment A include: Element A1 in combination with Element A2; Element A2 in combination with Element A3; Element A4 and/or Element A5 and optionally at least one of Elements A5-A6 in combination with at least one of Elements A1-A3; Element A8 in combination with at least one of Elements A1-A3; Element A9 in combination with at least one of Elements A1-A3; Element A10 in combination with at least one of Elements A1-A3; Element A11 and optionally Element A10 in combination with at least one of Elements A1-A3; Element A10 in combination with Element A11; and Element A10, Element A11, or both in combination with Element A4 and/or Element A5 and optionally at least one of Elements A5-A6.

Embodiment B is a method that includes conveying an NMR tool through a wellbore penetrating a subterranean formation containing a crude oil having an unknown concentration of asphaltene; measuring a first NMR property of the crude oil in the subterranean formation, wherein the first NMR property is T2, T1, a CMPG echo train, or any combination thereof; transmitting the first NMR property to a processor; and applying the first NMR property to a correlation for asphaltene concentration in crude oil as a function of a second NMR property so as to determine an asphaltene concentration in the crude oil having the unknown concentration of asphaltene, wherein the second NMR property is the T2, the T1, the CMPG echo train, or any combination thereof.

Embodiment B may have one or more of the following additional elements in any combination: Element B1: wherein the correlation is a mathematical regression according to Equation 3 and the plurality of crude oil samples having the known concentration of asphaltene have an API of about 20 to about 41, wherein C is an asphaltene concentration in the plurality of crude oil samples having the known concentration of asphaltene, k is a Huggins constant that describes solvent quality of the plurality of crude oil samples having the known concentration of asphaltene, and [η] is an intrinsic viscosity of the plurality of crude oil samples having the known concentration of asphaltene; Element B2: wherein the first NMR property and the second NMR property are different; Element B3: wherein the plurality of crude oil samples having the known concentration of asphaltene have an API gravity of about 20 to about 41; Element B4: the method further including treating the crude oil in the subterranean formation with a chemical to reduce asphaltene precipitation; and producing the crude oil in the subterranean formation; Element B5: the method further including treating the crude oil in the subterranean formation with steam; and producing the crude oil in the subterranean formation; Element B6: the method further including producing the crude oil from a subterranean formation using a system that includes a wellhead; and treating the crude oil at or near a wellhead with a chemical to reduce asphaltene precipitation; and Element B7: the method further including producing the crude oil from a subterranean formation using a system that includes a wellhead; and changing a temperature at the wellhead to reduce asphaltene precipitation; Element B8: wherein the NMR tool is coupled to or otherwise a portion of a drill string and the method further includes drilling the wellbore; and Element B9: wherein the NMR tool is coupled to a wireline.

By way of non-limiting example, exemplary combinations applicable to Embodiment B include: Element B1 in combination with Element B2; Element B2 in combination with Element B3; at least two of Elements B4-B7 in combination; at least one of Elements B1-B3 in combination with at least one of Elements B4-B7; and Element B8 and/or Element B9 in combination with one or more of Elements B1-B7 including in the foregoing combinations.

Embodiment C is a system that includes a NMR tool; a processor communicably coupled to the NMR tool and including a first non-transitory, tangible, computer-readable storage medium: containing a first program of instructions that cause a first computer system running the first program of instructions to: receive measured data of a first NMR property from the NMR tool; apply a correlation for asphaltene concentration in crude oil as a function of a second NMR property so as to determine an asphaltene concentration in a crude oil having an unknown concentration of asphaltene, wherein the second NMR property is the T1, the T2, the CMPG echo train, or any combination thereof.

Embodiment C may have one or more of the following additional elements in any combination: Element C1: wherein the correlation is a mathematical regression according to Equation 3 and the plurality of crude oil samples having the known concentration of asphaltene have an API of about 20 to about 41, wherein C is an asphaltene concentration in the plurality of crude oil samples having the known concentration of asphaltene, k is a Huggins constant that describes solvent quality of the plurality of crude oil samples having the known concentration of asphaltene, and [η] is an intrinsic viscosity of the plurality of crude oil samples having the known concentration of asphaltene; Element C2: wherein the first NMR property and the second NMR property are different; Element C3: wherein the plurality of crude oil samples having the known concentration of asphaltene have an API gravity of about 20 to about 41; Element C4: the system further including a drill bit attached to the distal end of a drill string, the drill string having the NMR tool coupled thereto or otherwise a portion thereof; and a pump operably connected to the drill string for circulating the drilling fluid through the drill string to an annulus defined by the drill string and the wellbore; Element C5: the system further including a wireline extending into a wellbore penetrating a subterranean formation with the NMR tool coupled to the wireline and disposed in the wellbore; Element C6: the system further including a wellhead at a surface location of a wellbore penetrating a subterranean formation with the NMR tool coupled to or otherwise a portion of the wellhead.

By way of non-limiting example, exemplary combinations applicable to Embodiment C include: at least two of Elements C1-C3 in combination and optionally in further combination with one or more of Elements C4-C6; and one of Elements C1-C3 in combination with one or more of Elements C4-C6.

To facilitate a better understanding of the embodiments of the present invention, the following examples of preferred or representative embodiments are given. In no way should the following examples be read to limit, or to define, the scope of the invention.

### Examples

Example 1 provides an example of experimentally obtaining

$$C ( \frac{1}{{T}_{1 }} , \frac{1}{{T}_{2}} ) .$$

Several crude oil samples from wellbores around the world were analyzed via NMR, specifically T1GM and T2GM (GM=geometric mean) were measured for each sample. The T2GM includes 5 different echo spacings (0.1, 0.4, 0.6, 0.9, and 1.2 ms), which show no appreciable difference. Each crude oil sample had an API specific gravity of about 20 to about 41. Further, the asphaltene concentration was determined by SARA analysis.

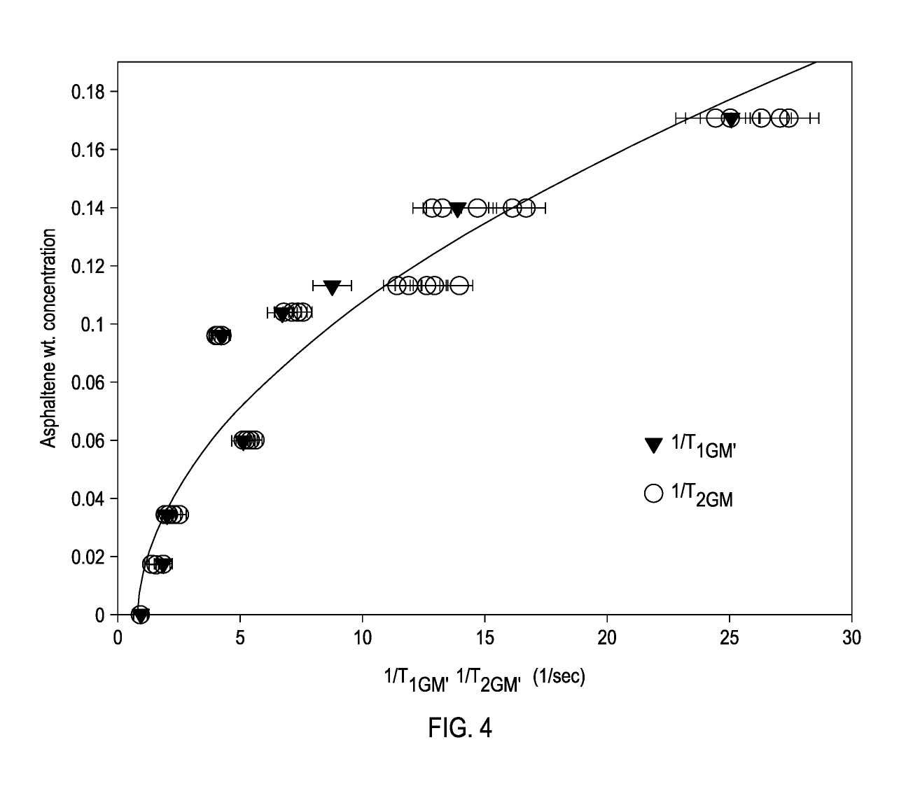

FIG. 4 illustrates the relationship between asphaltene concentration (weight fraction) versus 1/T1GM and 1/T2GM of the crude oil samples. Due to motional narrowing, it is observed that 1/T1GM 1/T2GM within the experimental uncertainties as indicated by the error bars, which also indicates that entanglement of solvent or maltene components due to the fractal nature of asphaltenes does not play a significant role.

In this example, it is assumed that

$$\frac{1}{{T}_{1}} = \frac{1}{{T}_{2}} ∼ 1 + [ η ] C + {k [ η ]}^{2} {C}^{2} ∼ \frac{1}{{T}_{1}} = \frac{1}{{T}_{2}} ∼ 1 + BC + {DC}^{2} .$$

Then, the equations are solved for concentration,

$$C ( \frac{1}{{T}_{1 }} , \frac{1}{{T}_{2}} )$$

where B and D are coefficients: B=0.34, and D=15.2 that can be related to k and [η], assuming [η]=10 and k=0.5.

This example demonstrates the feasibility of estimating the asphaltene and maltene weight fractions of crude oil (maltene wt. fraction=1−asphaltene wt. fraction) as a function of the total relaxation time for T1 and T2.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

The invention claimed is:

1. A method comprising:
determining a correlation for asphaltene concentration in crude oil as a function of a first nuclear magnetic resonance (NMR) property using a plurality of crude oil samples having a known concentration of asphaltene, wherein the first NMR property is a spin-spin relaxation (T2), a spin-lattice relaxation (T1), a Carr-Meiboom-Purcell-Gill (CMPG) echo train, or any combination thereof;
measuring a second NMR property of a crude oil sample having an unknown concentration of asphaltene, wherein the second NMR property includes the T1, the T2, the CMPG echo train, or any combination thereof; and
determining the asphaltene concentration of the crude oil sample having the unknown concentration of asphaltene by applying the second NMR property to the correlation.

2. The method of claim 1, wherein the correlation is a mathematical regression according to Equation 3 and the plurality of crude oil samples having the known concentration of asphaltene have an American Petroleum Institute (API) gravity of about 20 to about 41, wherein C is an asphaltene concentration in the plurality of crude oil samples having the known concentration of asphaltene, k is a Huggins constant that describes solvent quality of the plurality of crude oil samples having the known concentration of asphaltene, and [η] is an intrinsic viscosity of the plurality of crude oil samples having the known concentration of asphaltene
$$\frac{1}{{T}_{1}} = \frac{1}{{T}_{2}} ∼ 1 + [ η ] C + {k [ η ]}^{2} {C}^{2} . \qquad Equation 3$$

3. The method of claim 1, wherein the first NMR property and the second NMR property are different.

4. The method of claim 1 further comprising:
conveying an NMR tool via wireline through a wellbore penetrating a subterranean formation while measuring the second NMR property, wherein the crude oil sample having the unknown concentration of asphaltene is a crude oil in the subterranean formation surrounding the wellbore.

5. The method of claim 1 further comprising:
drilling a wellbore penetrating a subterranean formation while measuring the second NMR property, wherein the crude oil sample having the unknown concentration of asphaltene is a crude oil in the subterranean formation surrounding the wellbore.

6. The method of claim 5 further comprising:
treating a crude oil in the subterranean formation with a chemical to reduce asphaltene precipitation; and
producing the crude oil in the subterranean formation.

7. The method of claim 5 further comprising:
treating a crude oil in the subterranean formation with steam; and
producing the crude oil in the subterranean formation.

8. The method of claim 1, wherein the crude oil sample having the unknown concentration of asphaltene is a refinery feedstock.

9. The method of claim 1, wherein the crude oil sample having the unknown concentration of asphaltene is a first feedstock, the method further comprising: mixing the first feedstock with a second feedstock of crude oil with a known concentration of asphaltene in appropriate quantities to produce a refinery feedstock with a desired asphaltene concentration.

10. The method of claim 1 further comprising:
producing a crude oil from a subterranean formation using a system that includes a wellhead, wherein the crude oil sample having the unknown concentration of asphaltene is a portion of the crude oil;
then, measuring the second NMR property of the crude oil sample having the unknown concentration of asphaltene; and
treating the crude oil at or near a wellhead with a chemical to reduce asphaltene precipitation.

11. The method of claim 1 further comprising:
producing a crude oil from a subterranean formation using a system that includes a wellhead, wherein the crude oil sample having the unknown concentration of asphaltene is a portion of the crude oil;
then, measuring the second NMR property of the crude oil sample having the unknown concentration of asphaltene; and
changing a temperature at the wellhead to reduce asphaltene precipitation.

12. A method comprising:
conveying an NMR tool through a wellbore penetrating a subterranean formation containing a crude oil having an unknown concentration of asphaltene;
measuring a first nuclear magnetic resonance (NMR) property of the crude oil in the subterranean formation, wherein the first NMR property is a spin-spin relaxation (T2), a spin-lattice relaxation (T1), a Carr-Meiboom-Purcell-Gill (CMPG) echo train, or any combination thereof;
transmitting the first NMR property to a processor; and
applying the first NMR property to a correlation for asphaltene concentration in crude oil as a function of a second nuclear magnetic resonance (NMR) property so as to determine an asphaltene concentration in the crude oil having the unknown concentration of asphaltene, wherein the second NMR property is the T2, the T1, the CMPG echo train, or any combination thereof.

13. The method of claim 12, wherein the NMR tool is coupled to or otherwise a portion of a drill string and the method further includes drilling the wellbore.

14. The method of claim 12, wherein the correlation is a mathematical regression according to Equation 3 and the plurality of crude oil samples having the known concentration of asphaltene have an American Petroleum Institute (API) gravity of about 20 to about 41, wherein C is an asphaltene concentration in the plurality of crude oil samples having the known concentration of asphaltene, k is a Huggins constant that describes solvent quality of the plurality of crude oil samples having the known concentration of asphaltene, and [η] is an intrinsic viscosity of the plurality of crude oil samples having the known concentration of asphaltene
$$\frac{1}{{T}_{1}} = \frac{1}{{T}_{2}} ∼ 1 + [ η ] C + {k [ η ]}^{2} {C}^{2} . \qquad Equation 3$$

15. The method of claim 12 further comprising:
treating the crude oil in the subterranean formation with a chemical to reduce asphaltene precipitation; and
producing the crude oil in the subterranean formation.

16. The method of claim 12 further comprising:
treating the crude oil in the subterranean formation with steam; and
producing the crude oil in the subterranean formation.

17. The method of claim 12 further comprising:
producing the crude oil from a subterranean formation using a system that includes a wellhead; and
treating the crude oil at or near a wellhead with a chemical to reduce asphaltene precipitation.

18. The method of claim 12 further comprising:
producing the crude oil from a subterranean formation using a system that includes a wellhead; and
changing a temperature at the wellhead to reduce asphaltene precipitation.

19. The method of claim 12, wherein the first NMR property and the second NMR property are different.

20. A system comprising:
a nuclear magnetic resonance (NMR) tool;
a processor communicably coupled to the NMR tool and including a first non-transitory, tangible, computer-readable storage medium: containing a first program of instructions that cause a first computer system running the first program of instructions to:
receive measured data of a first NMR property from the NMR tool;
apply a correlation for asphaltene concentration in crude oil as a function of a second NMR property so as to determine an asphaltene concentration in a crude oil having an unknown concentration of asphaltene, wherein the second NMR property is the T1, the T2, the CMPG echo train, or any combination thereof.

21. The system of claim 20 further comprising:
a drill bit attached to the distal end of a drill string, the drill string having the NMR tool coupled thereto or otherwise a portion thereof; and
a pump operably connected to the drill string for circulating the drilling fluid through the drill string to an annulus defined by the drill string and the wellbore.

22. The system of claim 20 further comprising:
a wireline extending into a wellbore penetrating a subterranean formation with the NMR tool coupled to the wireline and disposed in the wellbore.

23. The system of claim 20 further comprising:
a wellhead at a surface location of a wellbore penetrating a subterranean formation with the NMR tool coupled to or otherwise a portion of the wellhead.
